(19) United States
(12) Patent Application Publication
(10) Pub. No.: US 2009/0053237 A1
(43) Pub. Date: Feb. 26, 2009

(54) USE OF PENICILLIN-BINDING PROTEINS OR POLYNUCLEOTIDES OR ANTIBODIES THEREOF FOR PREVENTING OR TREATING BACTERIAL INFECTIONS

(75) Inventors: Maria Leticia Zarantonelli, Paris (FR); Aude Antignac, Chateaugiron (FR); Jean-Michel Alonso, Montigny Le Bretonneux (FR); Muhamed-Kheir Taha, Chennevieres (FR); Faridabano Nato, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: INSTITUT PASTEUR, PARIS (FR)

(21) Appl. No.: 11/915,055

(22) PCT Filed: May 22, 2006
(86) PCT No.: PCT/EP2006/062509

§ 371 Date: Nov. 20, 2007

(30) Foreign Application Priority Data
May 20, 2005 (EP) 05291086.6

(51) Int. Cl. A61K 39/395; C07K 14/00; C07H 21/00; C07K 16/00; A61P 31/04; A61P 37/04; G01N 33/53; A61K 38/16; A61K 39/095

(52) U.S. Cl. 424/164.1; 530/350; 536/23.7; 530/387.1; 514/12; 424/250.1; 436/501; 530/388.4

(57) ABSTRACT

The present invention is drawn to a penicillin-binding protein expressed by Neisseria meningitidis, or a fragment, or a variant, or a variant fragment thereof, or nucleic acids encoding them, or antibodies or antibody fragments directed against them, as medicaments or in pharmaceutical compositions. The invention is also directed to the use of penicillin-binding proteins expressed by Neisseria meningitidis, or nucleic acids encoding them, or antibodies directed against them for the prophylactic and/or therapeutic immunotherapy of mammals, preferably human beings, against infection by Neisseria meningitidis and/or by a bacterial strain expressing a variant of a Neisseria meningitidis penicillin-binding protein.

The present invention is drawn to a penicillin-binding protein expressed by Neisseria meningitidis, or a fragment, or a variant, or a variant fragment thereof, or nucleic acids encoding them, or antibodies or antibody fragments directed against them, as medicaments or in pharmaceutical compositions. The invention is also directed to the use of penicillin-binding proteins expressed by Neisseria meningitidis, or nucleic acids encoding them, or antibodies directed against them for the prophylactic and/or therapeutic immunotherapy of mammals, preferably human beings, against infection by Neisseria meningitidis and/or by a bacterial strain expressing a variant of a Neisseria meningitidis penicillin-binding protein.

Two complementary therapeutic strategies have greatly improved the capacity to resist bacterial infections: the use of antibiotics and the prophylactic or therapeutic vaccination of exposed individuals. However, although the combination of these two strategies allowed to almost eradicate some bacterial diseases, some difficulties aroused concerning other infections. On the one hand, an increasing number of bacterial strains developed resistance to the most common antibiotics, creating an endless competition between the conception of new antibiotics and the adaptation of bacteria to these new antibiotics. On the other hand, the elaboration of vaccines against some bacteria came up against the difficulty to find an efficient vaccine target and the genetic variability of potential protein targets.

In particular, the management of meningococcal infections requires immediate antibiotic therapy for the case and also chemoprophylaxis and vaccination (when available) of household contacts to prevent secondary cases and epidemics. Penicillin G is the antibiotic of choice against meningococcal disease (Quagliarello, V. J., and W. M. Scheld. 1997. N Engl J Med 336:708-16) but meningococcal strains with diminished susceptibility are increasingly recorded (Antignac, A., et al. 2003. Clin Infect Dis 37:912-20; Saez Nieto, et al. 1990. Lancet 336:54).

Current vaccines against Neisseria meningitidis are based on capsular polysaccharide and are available only against strains belonging to serogroups A, C, Y and W135 but not against strains of serogroup B. Protein candidates have been explored to develop vaccines against strains of serogroup B such as the major outer membrane porin (PorA) and the transferrin binding protein B (TbpB) (Claassen, I., et al. 1996. Vaccine 14:1001-8; Rokbi, B., et al. 1997. Infect Immun 65:55-63). Outer membrane vesicles-based vaccines were also developed (Fredriksen, J. H. et al. 1991. NIPH Ann 14:67-80; Holst, J., et al. 2003. Vaccine 21:734-7; Sierra, G. V., et al. 1991. NIPH Ann 14:195-210). However, this strategy is hindered by the high degree of variability of Neisseiria meningitidis due to horizontal DNA exchanges between strains, resulting in modification of structures of genetic loci (Maiden, M. C. 1993. FEMS Microbiol Lett 112:243-50).

There is thus a need for new vaccine candidates against bacterial infections, in particular Neisseria meningitidis infection. An ideal vaccine candidate molecule should be present in all strains of a particular bacterial species and have conserved regions that can be targeted by protective antibodies.

Penicillin-binding proteins (PBPs) are conserved proteins that are involved in the assembly and metabolism of the bacterial cell wall peptidoglycan (Goffin, C., and J. M. Ghuysen. 1998. Microbiol Mol Biol Rev. 62(4):1079-93). PBPS are constituted of two functional modules: an acyl serine transferase penicillin-binding (PB) module, which is linked to the carboxy end of a non-penicillin-binding (n-PB) module through a conserved fusion site. The two modules form a single polypeptide chain which folds on the exterior of the plasma membrane and is anchored by a transmembrane spanner. PBPs cluster into two classes, A and B. In each class, several conserved amino acid patterns are present both in the PB and n-PB modules (FIG. 1). Outside these conserved amino acid patterns, a great variability can be observed among strains of several species such as Neisseria meningitidis and Streptococcus pneumoniae.

PBPs are also implicated in the mechanism of penicillin anti-bacterial action thanks to their ability to catalyze the rupture of the β-lactam amide bond of penicillin and the formation of a serine ester-linked penicilloyl enzyme, which is almost completely inert, thus unable to get involved in the biosynthesis of peptidoglycans. However, the use of β-lactam antibiotics resulted in the emergence and the spread of PBPs with a low or decreased affinity for the drug among important bacterial pathogens. This decreased affinity for penicillin is due to genetic alterations in the PB module (Goffin, C., and J. M. Ghuysen. Microbiol. Mol Biol Rev. 62(4):1079-93).

The implication of PBPs in the resistance to β-lactam antibiotics has been thoroughly investigated, but their possible role as vaccine candidates has not been systematically analyzed at the present time, in particular in Neisseiria meningitidis for which it has never been even suggested. Three PBPs (PBP1, PBP2 and PBP3) have been reported in Neisseiria meningitidis (Barbour, A. G. 1981. Antimicrob Agents Chemother 19:316-22) but others were identified based on the genome sequence (Parkhill, J., et al. 2000. Nature 404:502-6; Tettelin, H., et al. 2000. Science 287:1809-15). PBP2, encoded by penA gene, is a 60 kDa protein, which is homologous to high-molecular weight class B PBPs and most likely catalyzes a transpeptidation reaction that is necessary for the cross-linking of peptidoglycan in the meningococcal cell wall. Highly related penA^{S} and different penA^{I} alleles were described in susceptible strains (Pen^{S}) and strains with reduced susceptibility to penicillin G (Pen^{I}), respectively (Antignac, A., et al. 2001. J Antimicrob Chemother 47:285-96). While the 5′ half of penA is highly conserved, alterations in the 3′ half of the gene are responsible for reduced susceptibility to penicillin G and occur through DNA transformation (Spratt, B. G. 1994. Science 264:388-93). The active serine residue Sx₂K, the SxN and the KxG (usually KTG) motifs, which are all located in the 3′ transpeptidase-encoding region are usually conserved, but a high degree of polymorphism is observed in the surrounding sequences (Antignac, A., et al. 2003. J Biol Chem 278:31529-35; Antignac, A., et al. 2001. J Antimicrob Chemother 47:285-96).

Despite the high degree of polymorphism observed in the sequences surrounding the conserved active serine residue Sx₂K, the SxN and the KxG (usually KTG) motifs of Neisseria meningitidis PBP2, the inventors surprisingly found that this protein is immunogenic during Neisseria meningitidis human infection, and that immunization of mice with a PBP2 fragment induces anti-PBP2 antibodies.

In addition, antibodies directed against a fragment of the PBP2 protein of a particular Neisseria meningitidis strain are able to bind PBP2 proteins of other Neisseria meningitidis strains, including strains of distinct serogroups and/or with different levels of susceptibility to antibiotics.

Finally, the inventors also demonstrated that immunization of mice with a PBP2 fragment induces protective immunity in vaccinated mice, and that antibodies directed against a fragment of the PBP2 protein of a particular Neisseria meningitidis strain are able to protect mice from infection by the same Neisseria meningitidis strain or a mutant thereof expressing an altered PBP2 protein.

The present invention thus concerns a penicillin-binding protein expressed by Neisseria meningitidis, or a fragment, or a variant, or a variant fragment thereof, as a medicament.

By a “penicillin-binding protein (PBP)”, it is intended to designate a protein of the penicilloyl serine transferase family, comprising an acyl serine transferase penicillin-binding (PB) module linked to the carboxy end of a non-penicillin-binding (n-PB) module through a conserved fusion site, and a transmembrane spanner permitting the anchorage in the membrane. Three PBP proteins (PBP1, PBP2 and PBP3) have been reported in Neisseria meningitidis (Barbour, A. G. 1981. Antimicrob Agents Chemother 19:316-22). The analysis of the genomic sequence of two Neisseria meningitidis strains further suggests the existence of two additional PBP proteins in Neisseria meningitidis (Parkhill J. et al. 2000. Nature 404(6777):502-6; Tettelin H. et al. 2000. Science 287(5459):1809-15; which are herein incorporated by reference). All these PBP proteins are included in the scope of the present invention. References of amino acid sequences of Neisseria meningitidis PBP proteins expressed by particular Neisseria meningitidis strains are displayed in following Table 1. These sequences are particularly included in the scope of the present invention, but should not be considered as limiting the invention, since Neisseria meningitidis PBP proteins expressed by any other mutant Neisseria meningitidis strain are also included in the scope of the present invention.

TABLE 1
Particular amino acid sequences of Neisseria meningitidis PBP proteins

| | Accession number | | PB module | PB module |
|---|---|---|---|---|
| Name | (N. m. strain) | Sequence | position (aa) | conserved motifs |
| PBP1 | AAF42144 (MC58), | SEQ ID NO: 1, | 294-798 | STFK, SKN, KTG |
| | CAB83943 (Z2491) | SEQ ID NO: 2 | | |
| PBP2 | AAF40852 (MC58), | SEQ ID NO: 3, | 240-584 | SAIK, SSN, KTG |
| | CAB85289 (Z2491) | SEQ ID NO: 4 | | |
| PBP3 | AAF42135 (MC58) | SEQ ID NO: 5 | 114-469 | SIKK, SDN, KTG |
| PBP4 | AAF41162 (MC58), | SEQ ID NO: 6, | 31-312 | SISK, SEN, KTG |
| putative | CAB84232 (Z2491) | SEQ ID NO: 7 | | |
| Putative | AAF41288 (MC58) | SEQ ID NO: 8 | Not determined | Not determined |
| additional PBP | | | | |

A “fragment” of a penicillin-binding protein of Neisseria meningitidis means a partial penicillin-binding protein, which has the capacity, upon immunization with said fragment, to generate protective immunity, preferably protective antibodies, against Neisseria meningitidis infection, or against infections by bacteria expressing a PBP protein comprising said fragment or a variant thereof. A PBP fragment according to the invention thus comprises at least a T cell or B cell epitope, preferably a B cell epitope, and is thus preferably at least 6, at least 8, at least 10, more preferably at least 12, at least 15, at least 20, at least 25, at least 30, at least 40, at least 50, at least 75, at least 100 amino acids long. In certain embodiments, said fragment comprises at least one of the 3 conserved amino acid motifs of the PB module (Sx₂K, SxN, and KxG) of said PBP protein, preferably 2 or 3 of the conserved amino acid motifs of the PB module. Said fragment may comprise the complete PB module of said penicillin-binding protein. In particular, the fragment may be a partial Neisseria meningitidis penicillin-binding protein lacking the N-terminal transmembrane domain, in particular a partial Neisseria meningitidis PBP2 protein lacking the N-terminal transmembrane domain.

The term “variant” of a penicillin-binding protein expressed by Neisseria meningitidis refers to a penicillin-binding protein, which may be modified in one or more amino acid positions, and which has the following features:
(i) It has at least 25%, at least 30%, at least 40%, at least 50%, more preferably at least 60%, at least 70%, at least 80%, most preferably at least 90%, at least 95%, at least 97%, at least 98%, at least 99%, at least 99.5%, at least 99.9% identity with the PB module of the specifically described Neisseria meningitidis protein, and
(ii) Its conserved (Sx₂K, SxN, KxG) motifs have at least 70%, at least 80%, at least 90%, or 100% of identity with conserved amino acid motifs (Sx₂K, SxN, and KxG) of the specifically described Neisseria meningitidis protein.

According to the invention, a variant of a Neisseria meningitidis PBP protein has the capacity, upon immunization with said variant, to generate protective immunity, preferably protective antibodies, against Neisseria meningitidis infection, or against infection by a bacterial strain expressing said variant. Such a variant may also have the capacity, upon immunization with said variant, to generate protective immunity, preferably protective antibodies, against infection by a bacterial strain expressing another variant of said Neisseria meningitidis PBP protein.

Conserved motifs of most bacterial penicillin-binding proteins are well determined (see Goffin, C., and J. M. Ghuysen. Microbiol. Mol Biol Rev. 62(4):1079-93 for amino acid positions and accession numbers). In case of a putative penicillin-binding protein, conserved motifs are preferably determined so that the Sx2K and SxN motifs are separated by at least 45 amino acids and the SxN and KxG (usually KTG) motifs are separated by at least 100 amino acids.

Preferably, a variant, as defined above, which has an identity with the PB module of the specifically described Neisseria meningitidis protein inferior to 50%, additionally has a spatial three-dimensional (3D) structure that is similar to that of the specifically described Neisseria meningitidis protein.

A “percentage of sequence identity” is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

The definition of sequence identity given above is the definition that would use one of skill in the art. The definition by itself does not need the help of any algorithm, said algorithms being helpful only to achieve the optimal alignments of sequences, rather than the calculation of sequence identity. From the definition given above, it follows that there is only one well defined value for the sequence identity between two compared sequences, which value corresponds to the value obtained for the best or optimal alignment.

The percentage of sequence identity between 2 amino acid sequences may be determined by using with default parameters the Blastp 2.0 program provided by the National Center for Biotechnology Information (http://www.ncbi.nlm.nih.gov/blast/; Tatusova et al., “Blast 2 sequences—a new tool for comparing protein and nucleotide sequences”, FEMS Microbiol. Lett. 174: 247-250), which is habitually used by the inventors and in general by the skilled man for comparing and determining the identity between two sequences.

The “three-dimensional (3D) structure similarity” between a Neisseria meningitidis PBP protein and a putative variant can be evaluated according to the goodness of fit between the two polypeptides. Goodness of fit can be represented by a variety of parameters known in the art including, for example, the root mean square deviation (RMSD). A lower RMSD between structures correlates with a better fit compared to a higher RMSD between structures (see for example, Doucet and Weber, Computer-Aided Molecular Design: Theory and Applications, Academic Press, San Diego, Calif. (1996)). Polypeptides with a similar three-dimensional (3D) structure can be identified by comparing mean RMSD values for the backbones of the polypeptides. Preferably, a Neisseria meningitidis PBP protein and a variant thereof can have a mean backbone RMSD compared to each other that is less than about 5A or less than about 3A.

Particular examples of variants of Neisseria meningitidis PBP proteins that are particularly included in the scope of the invention are displayed in following Table 2, as well as references of amino acid sequences of these variants in particular bacterial strains. Once again, these sequences are particularly included in the scope of the present invention, but should not be considered as limiting the invention, since any other variant, as defined above, of a Neisseria meningitidis PBP protein is also included in the scope of the present invention.

TABLE 2
Particular amino acid sequences of several Neisseria meningitidis PBP proteins
variants

| | | | | PB | | | | |
|---|---|---|---|---|---|---|---|---|
| | | Accession | | module | PB module | N.m. | % identity | % identity with |
| | | number | | position | conserved | closer | with PB | (Sx₂K, SxN, and KTG) |
| Name | Species | (strain) | Sequence | (aa) | motifs | PBP | module | motifs |
| Ngo- | Neisseria | AAW88860 | SEQ ID NO:9 | 294-798 | STFK, SKN, KTG | PBP1 | 93 | 100 |
| PBP1 | gonorrhoeae | (FA 1090) | | | | | | |
| Spn | Streptococcus | CAA05302 | SEQ ID NO:10 | 395-821 | STTK, SWN, KTG | PBP1 | 30 | 80 |
| PBP1b | pneumoniae | (R6) | | | | | | |
| Spn- | Streptococcus | CAA05303 | SEQ ID NO:11 | 340-731 | STIK, SLN, KTG | PBP1 | 30 | 80 |
| PBP2a | pneumoniae | (R6) | | | | | | |
| Ngo- | Neisseria | AAW90178 | SEQ ID NO:12 | 240-582 | SAIK, SSN, KTG | PBP2 | 99 | 100 |
| PBP2 | gonorrhoeae | (FA 1090) | | | | | | |
| Spn- | Streptococcus | AAK99108 | SEQ ID NO:13 | 266-750 | STMK, SSN, KSG | PBP2 | 27 | 70 |
| PBP2X | pneumoniae | (R6) | | | | | | |

According to the invention, a “variant fragment” of a Neisseria meningitidis PBP protein is a fragment, as defined above, of a variant, as defined above, which further has at least 25%, at least 30%, at least 40%, at least 50%, more preferably at least 60%, at least 70%, at least 80%, most preferably at least 90%, at least 95%, at least 97%, at least 98%, at least 99%, at least 99.5%, at least 99.9% identity with the corresponding Neisseria meningitidis PBP protein fragment.

According to the invention, such a penicillin-binding protein expressed by Neisseria meningitidis, or variant thereof, may or may not be implicated in β-lactam antibiotics resistance. Examples of β-lactam antibiotics to which bacterial strains may become resistant due to the expression of new or mutated penicillin-binding proteins include penicillin G, amoxicillin, ampicillin, and cephalosporins.

According to the invention, a PBP protein expressed by Neisseria meningitidis, or fragment, or variant or variant fragment thereof may be naturally occurring or man-made by peptide synthesis or conventional recombinant technologies. In particular, it may or may not display any of the posttranslational modifications displayed by a naturally occurring Neisseria meningitidis PBP protein, or fragment, or variant, or variant fragment thereof, such as glycosylation patterns. According to the invention, a Neisseria meningitidis PBP protein, or fragment, or variant or variant fragment thus include any polypeptide with the amino acid sequence of a Neisseria meningitidis PBP protein, or fragment, or variant, or variant fragment thereof.

It may be further modified to facilitate its production, purification, or to improve its stability, biodisponibility or efficiency as a medicament. For example, it may be coupled to a carbohydrate, a lipid moiety or modified in other ways, e.g. phosphorylated or hydroxylated. In particular, it may be glycosylated, the coupled carbohydrate moiety having molecular weights of 20-30 kD in the natural molecule. Coupling of said Neisseria meningitidis PBP, or fragment, or variant, or variant fragment to one or more moieties may for instance be due to a posttranslational modification of the polypeptide performed by an organism producing the polypeptide or a modification resulting from chemical synthesis.

Said PBP expressed by Neisseria meningitidis, or fragment, or variant, or variant fragment may also be fused to another polypeptide sequence, which may be one that results in an increased expression of the protein when expressed in an organism, or facilitates or improves the purification and recovery of the fusion protein from said organism in terms of a more easy and economical recovery, or improves the immunogenicity or pharmacokinetics of said Neisseria meningitidis PBP, or fragment, or variant, or variant fragment thereof.

In some cases, it may be advantageous to cleave the fusion protein so as to obtain a polypeptide which substantially solely comprises said Neisseria meningitidis PBP, or fragment, or variant, or variant fragment. In these cases, said Neisseria meningitidis PBP, or fragment, or variant, or variant fragment thereof is preferably fused to a polypeptide sequence which may be specifically recognized by a cleaving agent, e.g. a chemical such as cyanogen bromide, hydroxylamine and 2-nitro-5-thiocyanobenzoate, or an enzyme, e.g. a peptidase, proteinase or protease, e.g. trypsin, chlostripain, and staphylococcal protease or factor Xa

The present invention also concerns a nucleic acid encoding at least one penicillin-binding protein expressed by Neisseria meningitidis, or a fragment, or a variant, or a variant fragment thereof, as a medicament.

According to the invention, a nucleic acid encoding at least one penicillin-binding protein expressed by Neisseria meningitidis, or fragment, or variant, or variant fragment thereof, may be any kind of nucleic acid, including single-stranded DNA, double-stranded DNA, single-stranded RNA, double-stranded RNA, or a RNA/DNA hybrib. In a preferred embodiment, the nucleic acid is either double-stranded DNA or single-stranded RNA. In another embodiment, the nucleic acid is an antisens RNA.

Such a nucleic acid may encode either a single PBP protein expressed by Neisseria meningitidis or a fragment, or a variant, or a variant fragment thereof, or multiple distinct PBP proteins expressed by Neisseria meningitidis or fragments, or variant, or variants fragments thereof. It may further encode other polypeptides. In the nucleic acid, at least one encoded Neisseria meningitidis PBP or variant thereof may be fused to any other polypeptide.

In the case of DNA, said nucleic acid may be provided with operably linked start and stop codons, promoter and terminator regions, and optionally other regulating sequences, such as an enhancer. The promoter, and optionally the enhancer allow for the expression of the PBP, or fragment or variant or variant fragment thereof coding sequence in a host target cell, which may be a prokaryotic or eukaryotic host cell. In one preferred embodiment, the DNA nucleic acid includes a cell-specific promoter that permits substantial transcription of the DNA only in predetermined cells. The DNA nucleic acid may be engineered so as not to integrate into the host cell genome.

Such a DNA nucleic acid may be inserted into a plasmid of partly bacterial origin, and notably carrying a bacterial replication origin and a gene allowing its selection, such as a gene for resistance to an antibiotic. This plasmid may also be provided with a replication origin allowing it to replicate in the target cells of its host, for instance, in the case of muscle cells, the replication origin of the bovine papilloma virus. Many such plasmids are known from one skilled in the art and a great number are commercially available.

A nucleic acid according to the invention may be naked or inserted into any delivery system know by one skilled in the art. For instance, delivery systems include viral vectors, bacterial vectors, or chemical vectors. Examples of suitable viral vectors include herpes simplex viral vectors, vaccina or alpha-virus vectors and retroviruses, including lentiviruses, adenoviruses and adeno-associated viruses. Preferably, these vectors are replication defective virus vectors.

Examples of chemical vectors are liposomes, or oil-in-water emulsions. Alternatively, a nucleic acid according to the invention may be attached to at least one carrier molecule. Such a carrier molecule may be a molecule that facilitates the targeting of the nucleic acid to a particular cell type, in particular a ligand capable of binding to the surface of a target cell, such as a ligand of a membrane receptor or an antibody directed against a membrane receptor.

Alternatively, a carrier molecule according to the invention may be a nucleic binding molecule preferably capable to protect the nucleic acid against nuclease enzymes. For instance, a nucleic binding molecule may be a polycationic polymer, such as poly-L-lysine, poly-D-lysine, polyethyleneimine, polyamidoamine, or polyamine; or a nucleic binding protein, such as a histone protein, a protamine, ornithine, putrescine, spermidine, spermine, a transcription factor or a homeobox protein.

Still another type of carrier molecule may be a polypeptide that is able to direct the intracellular localisation of the nucleic acid in a host cell. For instance, it may be a signal polypeptide that directs the nucleic acid to the nucleus in an eukaryote host cell.

The present invention also concerns an antibody or an antibody fragment directed against a penicillin-binding protein expressed by Neisseria meningitidis or a fragment, or a variant, or a variant fragment thereof, as a medicament.

The term “antibody” includes a polypeptide encoded by an immunoglobulin gene or immunoglobulin genes, capable of specifically binding to an antigen or epitope, and expressed by a B lymphocyte or a B lymphocyte derived clone. The term “antibody” is used in the broadest sense. Therefore, an “antibody” can be naturally occurring or man-made by peptide synthesis or by the production of monoclonal antibodies by conventional hybridoma technology.

An “antibody fragment” is defined as at least a portion of the variable region of the immunoglobulin molecule that binds to its target, i.e., the antigen-binding region. Well-known “antibody fragments” include:
(i) a Fab fragment: a monovalent fragment consisting of the VL, VH, CL and CHI domains;
(ii) a F(ab′)2 fragment: a bivalent fragment comprising two Fab fragments linked by a disulfide bridge at the hinge region;
(iii) a Fd fragment consisting of the VH and CHI domains;
(iv) a Fv fragment consisting of the VL and VH domains of a single arm of an antibody;
(v) a dAb fragment (Ward et al., (1989) Nature 341:544-546), which consists of a VH domain; and
(vi) an isolated complementary determining region (CDR).

Single chain antibodies are also included by reference in the term “antibody fragment”.

Methods of immunization, producing and isolating antibodies (polyclonal and monoclonal) are known to those skilled in the art and described in the scientific and patent literature. Antibodies also can be generated in vitro, e.g., using recombinant antibody binding site expressing phage display libraries, in addition to the traditional in vivo methods using animals.

Said antibodies or antibody fragments may belong to any class of antibody, such as IgM, IgD, IgG, IgE or IgA antibodies, as well as all subclasses such as IgG1, IgG2, IgG3, IgG4.

Said antibody or antibody fragment may be polyclonal or monoclonal, chimeric or humanized.

As used herein, the term “chimeric” applied to an antibody or an antibody fragment means that two or more segments or portions of said antibody or antibody fragment derive from different animal species. A particular case of chimeric antibody is a “humanized” antibody, which comprises human framework regions with complementary determining regions (CDR) of another species. Technologies for the humanization of antibodies from non-human species are well-known for the one skilled in the art.

In a preferred embodiment of a medicament constituted of a penicillin-binding protein, or fragment, or variant, or variant fragment thereof; or a nucleic acid; or an antibody or antibody fragment according to the invention, said penicillin-binding protein is Neisseria meningitidis PBP2.

In a particular embodiment, said penicillin-binding protein fragment is a fragment of Neisseria meningitidis PBP2 lacking the N-terminal transmembrane domain. In still a more particular embodiment, said fragment of Neisseria meningitidis PBP2 lacking the N-terminal transmembrane domain is purified from clone DH5pAA2 deposited on May 3, 2005 at the CNCM (25-28 rue du Dr Roux. 75015 Paris, France) under number I-3422. The DH5pAA2 clone was obtained by cloning the penA gene of Neisseria meningitidis LNP8013 strain, deleted of the first nucleotides coding for the first 41 amino acids, between the Nde I and Xho I sites of the pET28b vector (Antignac A. et al. 2003. J Biol. Chem. 278(34):31521-8).

In another preferred embodiment of a medicament constituted of a penicillin-binding protein, or fragment, or variant, or variant fragment thereof; or a nucleic acid; or an antibody or antibody fragment according to the invention, said variant is a variant of a Neisseria meningitidis PBP expressed by Neisseria gonorrhoeae or Streptococcus pneumoniae. Such variants of a Neisseria meningitidis PBP expressed by Neisseria gonorrhoeae or Streptococcus pneumoniae include Neisseria gonorrhoeae PBP1 and PBP2, and Streptococcus pneumoniae PBP1b, PBP2a, and PBP2X. Preferably, said variant is Neisseria gonorrhoeae PBP2 or Streptococcus pneumoniae PBP2X.

An antibody or antibody fragment medicament according to the invention may be used alone or in combination with other drugs. For instance, it may be used in combination with an antibiotic, said antibiotic being preferably chosen in the group of β-lactam antibiotics, in particular penicillin G, amoxicillin, ampicillin, and cephalosporins.

In addition, a medicament constituted of a penicillin-binding protein, or fragment, or variant, or variant fragment thereof; or a nucleic acid encoding them according to the invention may also be used alone or in combination with other drugs. Notably, it may be used in combination with an antibiotic. Examples of suitable antibiotics include rifampicin, spiramycin, quinolons and third generation cephalosporins, in particular cefotaxim.

The present invention further concerns a pharmaceutical composition comprising at least one penicillin-binding protein, or a fragment, or a variant, or a variant fragment thereof, or a nucleic acid, or an antibody or an antibody fragment according to the invention, with a pharmaceutically acceptable carrier or excipient. In the pharmaceutical composition, the penicillin-binding protein, or fragment, or variant, or variant fragment thereof, or the nucleic acid, or the antibody or antibody fragment may display any of the features previously described for a penicillin-binding protein, or nucleic acid, or antibody as a medicament according to the invention.

“Pharmaceutically acceptable” refers to a non-toxic, inert composition that is physiologically compatible with humans or other mammals.

Pharmaceutically acceptable carriers or excipients include dispersion agents, suspension agents, diluents, fillers, pH-adjusting and buffering agents, tonicity adjusting agents, wetting agents, preservatives, and the like.

The pharmaceutical compositions according to the invention may further comprise an adjuvant. The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. A number of adjuvants and putative adjuvants (such as certain cytokines) share the ability to enhance the immune response. Suitable cytokines to be used as adjuvants are those which will normally also function as adjuvants in a vaccine composition, e.g. interferon γ (IFN-γ), Flt3 ligand (Flt3L), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF). In the case of a medicament or a pharmaceutical composition comprising at least one nucleic acid encoding at least one PBP protein or fragment or variant or variant fragment thereof, said cytokine may also be encoded in the nucleic acid. Alternatively, the adjuvant may be a toxin, such as listeriolysin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities. Heat shock proteins may also be used as adjuvant, such as HSP70, HSP90, HSC70, GRP94, and calreticulin (CRT). In the case of a medicament or a pharmaceutical composition comprising at least one nucleic acid encoding at least one PBP protein or fragment or variant or variant fragment thereof, said heat shock protein may also be encoded in the nucleic acid.

Above-mentioned pharmaceutical compositions or medicaments may be administered by any route, including, but not limited to, oral, subcutaneous, intravenous, intranasal, transdermal, intraperitoneal, intramuscular, intrapulmonar, vaginal, rectal, or in any other acceptable manner.

Above-mentioned pharmaceutical compositions or medicaments may be formulated in a variety of forms, e.g. as a liquid, gel, lyophilized, or as a compressed solid. The preferred form will depend upon the particular indication being treated and will be apparent to one skilled in the art. In each case, the active ingredient or the combination of active ingredients to be administered should be formulated in a manner which will protect the active ingredient against degradation, in particular by enzymes.

Above-mentioned pharmaceutical compositions or medicaments may be co-administered, concurrently administered, and/or sequentially administered with one or more other therapeutic agents.

Above-mentioned pharmaceutical compositions or medicaments contain a therapeutically effective dose of active ingredient (i.e. at least one PBP or fragment or variant or variant fragment thereof, or at least one nucleic acid encoding at least one PBP or fragment or variant or variant fragment thereof, or at least one antibody or antibody fragment directed against at least one PBP or fragment or variant or variant fragment thereof). By “therapeutically effective dose” herein is meant a dose that is sufficient to produce the desired effects in relation to the condition for which it is administered. The exact dose will depend on the circumstances (the disease, the administration schedule, whether the pharmaceutical composition or medicament is administered alone or in conjunction with other therapeutic agents, the plasma half-life of the pharmaceutical composition or medicament, and the general health of the patient), and will be ascertainable by one skilled in the art using known techniques.

The present invention further concerns the use of at least one penicillin-binding protein or fragment, or variant, or variant fragment thereof according to the invention, for the preparation of a pharmaceutical composition for the vaccination of a mammal, preferably a human being, against infection by a bacterial strain expressing a Neisseria meningitidis PBP protein or a variant thereof. In such a use, the penicillin-binding protein, or fragment, or variant, or variant fragment thereof may display any of the features previously described for a penicillin-binding protein or fragment, or variant, or variant fragment thereof as a medicament according to the invention.

The invention also concerns the use of at least one nucleic acid according to the invention, for the preparation of a pharmaceutical composition for the vaccination of a mammal, preferably a human being, against infection by a bacterial strain expressing a Neisseria meningitidis PBP protein or a variant thereof. In such a use, said nucleic acid may display any of the features previously described for a nucleic acid as a medicament according to the invention.

By “vaccination” of a mammal against infection by a bacterial strain is meant an immunization of said mammal inducing protective immunity against said bacterial strain. By “immunization” is meant the stimulation of the immune system of said mammal, including the stimulation of the innate immune system, such as the complement system, dendritic cells, macrophages, neutrophils, eosinophils, and/or the adaptive immune system, such as T and B lymphocytes. The induction of “protective immunity” against infection by a bacterial strain means that the stimulation of the immune system induced by the immunization results in a state of immune resistance to said infectious bacterial strain. Said state of immune resistance to the infectious bacterial strain appears a few days after immunization and then persists for extended periods of time. The induction of protective immunity is thus useful in an already infected individual to help to jugulate the infection, or in a healthy individual to prevent a potential subsequent infection.

By “state of immune resistance”, it is meant that the extent of the infection is significantly diminished when compared to a control mammal that did not receive said pharmaceutical composition. The extent of the bacterial infection may be determined by measuring bacteremia (cfu counts) by well-known technologies in presumably infected organs at several time points after infection. Preferably, the term “state of immune resistance” is used when the extent of bacterial infection is significantly reduced when compared to a control mammal that did not receive said pharmaceutical composition. Preferably, the extent of bacterial infection at 3-5 hours after infection is reduced of at least a 200-300 factor when compared to a control mammal that did not receive said pharmaceutical composition. Such a state of immune resistance may be induced for instance when the vaccination induces antibodies directed against at least one penicillin-binding protein expressed by Neisseria meningitidis or a variant thereof. Such antibodies include all classes of antibody, such as IgM, IgD, IgG, IgE ant IgA antibodies, as well as all subclasses such as IgG1, IgG2, IgG3, IgG4.

In all the description, the term “mammal” refers to any organism classified as a mammal, including mice, rats, rabbits, dogs, cats, cows, horses and humans. In one embodiment of the invention, the mammal is a mouse. In another embodiment of the invention, the mammal is a human.

The invention also concerns the use of at least one antibody or antibody fragment according to the invention, for the preparation of a pharmaceutical composition for the protection of a mammal, preferably a human being, against infection by a bacterial strain expressing a Neisseria meningitidis PBP protein or a variant thereof. In such a use, said antibody or antibody fragment may display any of the features previously described for an antibody or antibody fragment as a medicament according to the invention.

By “protection”, it is meant that the extent of the bacterial infection is significantly diminished by the use of said pharmaceutical composition comprising one or several antibodies, or antibody fragments, when compared to a control mammal that did not receive said pharmaceutical composition. Preferably, the term “protection” is used when the extent of bacterial infection is significantly reduced when compared to a control mammal that did not receive said pharmaceutical composition. Preferably, the extent of bacterial infection at 3-5 hours after infection is reduced of at least a 200-300 factor when compared to a control mammal that did not receive said pharmaceutical composition.

In a preferred embodiment of a use according to the invention, said infectious bacterial strain is Neisseria meningitidis. In this case, said used pharmaceutical composition preferably vaccinates or protects said mammal against infection by all Neisseria meningitidis serogroups.

By “serogroup”, it is meant a group of related microorganisms distinguished by its composition of antigens. More precisely, Neisseria meningitidis serogroups are defined according to their capsule antigen composition, which is determined using antibodies directed to capsule antigens. Neisseria meningitidis strains are divided into five main serogroups: A, B, C, Y and W135. Current vaccines against Neisseria meningitidis are based on capsular polysaccharide and are available only against strains belonging to serogroups A, C, Y and W135 but not against strains of serogroup B. In contrast, by a use according to the invention, it is possible to vaccinate or protect a mammal against all Neisseria meningitidis serogroups.

In another preferred embodiment of a use according to the invention, said infectious bacterial strain is a strain expressing a variant of a Neisseria meningitidis PBP protein, such as Neisseria gonorrhoeae and Streptococcus pneumonia.

In another preferred embodiment, when the infectious bacterial strain is Neisseria meningitidis, said pharmaceutical composition further vaccinates or protects said mammal against infection by a bacterial strain of a distinct species or genus expressing a variant of a Neisseria meningitidis PBP protein.

Preferably, such a bacterial strain of a distinct species or genus expressing a variant of a Neisseria meningitidis PBP protein is Neisseria gonorrhoeae or Streptococcus pneumoniae.

In a particular embodiment, the use as described above of at least one penicillin-binding protein or fragment, or variant, or variant fragment thereof, or of at least one nucleic acid according to the invention, for the preparation of a pharmaceutical composition for the vaccination of a mammal, preferably a human being, against infection by a bacterial strain expressing a Neisseria meningitidis PBP protein or a variant thereof, is performed in combination with an antibiotic. Examples of suitable antibiotics include rifampicin, spiramycin, quinolons and third generation cephalosporins, in particular cefotaxim. The features of said bacterial strain expressing a Neisseria meningitidis PBP protein or variant thereof may vary according to any particular features described above.

In another particular embodiment, the use as described above of at least one antibody or antibody fragment for the preparation of a pharmaceutical composition for the protection of a mammal, preferably a human being, against infection by a bacterial strain expressing a Neisseria meningitidis PBP protein or a variant thereof, is performed in combination with an antibiotic, preferably a β-lactam antibiotic. Examples of suitable β-lactam antibiotics include penicillin G, amoxicillin, ampicillin, and cephalosporins. The features of said bacterial strain expressing a Neisseria meningitidis PBP protein or variant thereof may vary according to any particular features described above.

The invention also concerns the use of at least one penicillin-binding protein, or fragment, or variant or variant fragment thereof according to the invention, for the detection of antibodies directed against Neisseria meningitidis in a mammal, preferably a human being. In such a use, said penicillin-binding protein may display any of the features previously described for a penicillin-binding protein as a medicament according to the invention. Such a detection of antibodies directed against Neisseria meningitidis may be useful for instance to diagnose in vitro a Neisseria meningitidis infection in a non-vaccinated mammal, or to evaluate the efficiency of vaccination in a vaccinated mammal.

The detection may be performed using any of the many technologies known in the art for the detection of specific antibodies in serum using their purified specific antigen, such as for instance the ELISA technology. The advantage of the use of at least one penicillin-binding protein, or fragment, or variant or variant fragment thereof according to the invention is that it allows for the in vitro detection of antibodies directed against a Neisseria meningitidis strain of any serogroup.

The invention also concerns a method of in vitro detection from a biological sample of a mammal, preferably a human being, of antibodies directed against Neisseria meningitidis infection, comprising:
contacting said biological sample with at least one Neisseria meningitidis penicillin-binding protein, or fragment, or variant or variant fragment thereof, as described above, and
detecting the presence of complexes between said at least one Neisseria meningitidis penicillin-binding protein, or fragment, or variant or variant fragment thereof and an antibody of said biological sample.

Preferably, said biological sample is a blood sample, a serum sample or a plasma sample. As said above, the detection step may be performed using any of the many technologies known in the art for the detection of complexes between specific antibodies and their purified specific antigen, such as for instance the ELISA technology.

The invention further relates to the use of at least one antibody or antibody fragment according to the invention, for the in vitro diagnosis from a biological sample of a mammal, preferably a human being, of Neisseria meningitidis infection. In such a use, said antibody or antibody fragment may display any of the features previously described for an antibody or antibody fragment as a medicament according to the invention.

The diagnosis may be performed using any of the many technologies known in the art for the detection of a specific antigen in serum using their antibodies or antibody fragments specific for this antigen, such as for instance the ELISA technology. The advantage of the use of at least one antibody or antibody fragment according to the invention is that it allows for the in vitro diagnosis of infection by a Neisseria meningitidis strain of any serogroup.

The invention also concerns a method of in vitro diagnosis from a biological sample of a mammal, preferably a human being, of Neisseria meningitidis infection, comprising:
contacting said biological sample with at least one antibody or antibody fragment directed against a penicillin-binding protein expressed by Neisseria meningitidis, or a variant, or a fragment, or a variant fragment thereof according to the invention as described above, and
detecting the presence of complexes between said at least one antibody or antibody fragment and a Neisseria meningitidis penicillin-binding protein, or fragment thereof from said biological sample.

Preferably, said biological sample is a blood sample, a serum sample or a plasma sample. As said above, the detection step may be performed using any of the many technologies known in the art for the detection of complexes between specific antibodies and their purified specific antigen, such as for instance the ELISA technology.

The invention also concerns a method of treatment of a mammal, preferably a human being, against Neisseria meningitidis infection and/or against infection by a bacterial strain expressing a Neisseria meningitidis PBP variant, comprising vaccinating said mammal against at least one penicillin-binding protein expressed by Neisseria meningitidis, or a variant thereof.

Vaccination may be performed by any technology known in the art, in particular by using a pharmaceutical composition comprising at least one penicillin-binding protein expressed by Neisseria meningitidis, or a fragment, or a variant, or a variant fragment thereof, or a nucleic acid encoding them, as described above.

The invention also concerns a method of treatment of a mammal, preferably a human being, against Neisseria meningitidis infection and/or against infection by a bacterial strain expressing a Neisseria meningitidis PBP variant, comprising injecting to said mammal an effective amount of a medicament or a pharmaceutical composition comprising at least one antibody or antibody fragment directed against at least one penicillin-binding protein expressed by Neisseria meningitidis or a fragment, or a variant, or a variant fragment thereof, as described above.

The invention further concerns a monoclonal antibody directed against Neisseria meningitidis PBP2 protein selected from the group constituted of monoclonal antibodies produced from hybridomas D2.4, D13.6 and 14.37, deposited at CNCM on May 22, 2006 under numbers I-3609, I-3610 and I-3611 respectively. Such a monoclonal antibody may be used as a medicament as described above, or for any therapeutic use described above. Alternatively, such a monoclonal antibody may be used for the in vitro diagnosis from a biological sample of a mammal, preferably a human being, of Neisseria meningitidis infection.

Having generally described this invention, a further understanding of characteristics and advantages of the invention can be obtained by reference to certain specific examples and figures which are provided herein for purposes of illustration only and are not intended to be limiting unless otherwise specified.

### DESCRIPTION OF THE DRAWINGS

FIG. 1. Molecular Organization of PBPs of high molecular mass of class A (PBP1a of E. coli, PBP1 of N. gonorrhoeae and Neisseiria meningitidis) and of class B (PBP3 of ′E. coli, PBP2 of N. gonorrhoeae and Neisseiria meningitidis). The conserved motifs (numbered from 1 to 9 in class A PBPs and from 1 to 7 in class B PBPs) as well as the distances between the motifs (number of amino acids) are represented (Goffin and Ghuysen, 1998. Microbiol. Mol. Biol. Rev. Dec.; 62(4):1079-93.)

FIG. 2: Specificity of serum of a rabbit immunized with PBP2 of Neisseria meningitidis strain LNP8013.

FIG. 2A. SDS-PAGE of solubilized whole cells from E. coli BL21(DE3) pLysS and eluted fractions containing purified His₆-tag-PBP2 protein of Neisseria meningitidis strain LNP8013. Solubilized cells were from E. coli BL21(DE3) pLysS harboring the expression vector alone (lane 1), the recombinant plasmid carrying the penA gene without IPTG induction (lane 2) and after IPTG induction (lane 3). The arrow shows the band corresponding to the overexpressed recombinant PBP2 lacking the N-terminal transmembrane domain (121 aminoacids). Lane 4 corresponds to the soluble fraction that was passed trough the nickel column and lanes 5 to 11 represent the eluted fractions containing purified PBP2. MM: Molecular weight marker.

FIG. 2B. Western blot showing the binding of one rabbit serum obtained after immunization with the PBP2 protein of Neisseria meningitidis strain LNP8013. Rabbit serum was tested on purified His₆-tag-proteins (PP) corresponding to meningococcal strains LNP8013, LNP17723, LNP16454, LNP18425 and LNP17041, as well as bacterial membrane extracts (BME) from two serogroup W135 isolates (strains LNP17592 and LNP16635). NmC: serogroup C Neisseiria meningitidis. NmB: serogroup B Neisseiria meningitidis. NmW135: serogroup W135 Neisseiria meningitidis. Pen^{S}: susceptible strain to penicillin G. Pen^{I}: strain with intermediate susceptibility to penicillin G.

FIG. 3: Immunogenicity of PBP2 during natural meningococcal infection.

FIG. 3A. IgG immune response against whole meningococcal cells (solid symbols) in pairs of sera from patients (P1 to P3) obtained at admission to hospital (acute sera) and 7-20 days after (convalescent sera). Patient P1 is a patient with confirmed meningococcal infection with a serogroup C Neisseiria meningitidis. Patient P2 is a patient with confirmed meningococcal infection with a serogroup B Neisseiria meningitidis. Patient P3 is a patient with no meningococcal infection. Open symbols represent the activity of each pair of sera against E. coli whole cells.

FIG. 3B. Dot blot analysis performed with the P1 pair of sera (acute and convalescent) and the control P3 serum (convalescent) against purified recombinant His6-tag-PBP2s corresponding to Pen^{S} (PBP2^{S}) and Pen^{I} (PBP2^{I}) meningococcal isolates belonging to different serogroups and genetic lineages. Four different dilutions of sera were tested (1:500 to 1:5,000). For each protein, two concentrations (300 and 150 ng) were spotted, except for PBP2 from strain LNP8013 where 100 and 50 ng were used. Meningococcal pilin was purified as previously described (11).

FIG. 4: Passive protection studies against meningococcal bacteremia in mice challenged intraperitoneally. The protective role of the anti-PBP2 IgG was evaluated after i.p challenge with LNP8013 Neisseiria meningitidis strain harboring a wild type penA^{S} allele and three penA^{I} isogenic mutants, TR-TH41, TR16454, and TR16504. Purified rabbit IgG against PBP2 or CrgA were used. The used strain/IgG pairs are indicated above each curve. Solid and open symbols represent results from untreated control mice (PBS) and mice treated with 30 μg of IgG, respectively. Data are means ±S.D. of groups of three mice per time point from two independent experiments. Student's t test was used to compare numbers of CFU (log₁₀) between groups of untreated and treated mice. * P= or <0.05.

FIG. 5: Anti-meningococcal protection obtained in mice immunized with the PBP2 protein of penicillin susceptible Neisseria meningitidis serogroup C strain LNP8013. Vaccinated (n=5) and control mice (n=4) were challenged by i.p. infection with 5×10⁵ cfu of the penicillin susceptible meningococcal strain LNP8013. After three hours, cfu counts were determined in blood by plating serial blood dilutions on GCB plates. Results were normalised according to the initial inocula. Individual vaccinated mice (black rhombs) and control mice (black circles) are represented. A student's t-test was applied to determine the significance (p<0.05) of the differences between both groups of mice.

FIG. 6: Specificity of the passive protection against meningococcal bacteraemia in mice. A. The protective role of the anti-PBP2 IgG was evaluated after i.p challenge with LNP8013 N. meningitidis strain harbouring a wild type penA^{S}. Purified anti-PBP2 IgG (30 μg) alone or with purified recombinant His₆-tag-PBP2 lacking the N-terminal transmembrane domain from the penicillin susceptible serogroup C strain LNP8013 (20 μg) were injected before the bacterial challenge. Two hours later, mice were challenged i.p. with 3×10⁶ cfu of the bacterial suspension in 0.5 mL of PBS. Blood cfu counts from four mice were performed 4h after the bacterial challenge. Data are means ±SD of groups of 4 mice per time point from two independent experiments. B. Western blot analysis using purified anti-PBP2 IgG against the purified recombinant His6-tag-PBP2 lacking the N-terminal transmembrane domain from the penicillin susceptible serogroup C strain LNP8013 preparation used in A.

FIG. 7: The binding capacities of anti-PBP2 monoclonal antibodies (D2.4, D13.6 and 14.37) were tested by western blot. All three monoclonal antibodies recognized purified recombinants PBP2s from several different meningococcal strains belonging to serogroups B, C or W135 and with different levels of susceptibility to penicillin.

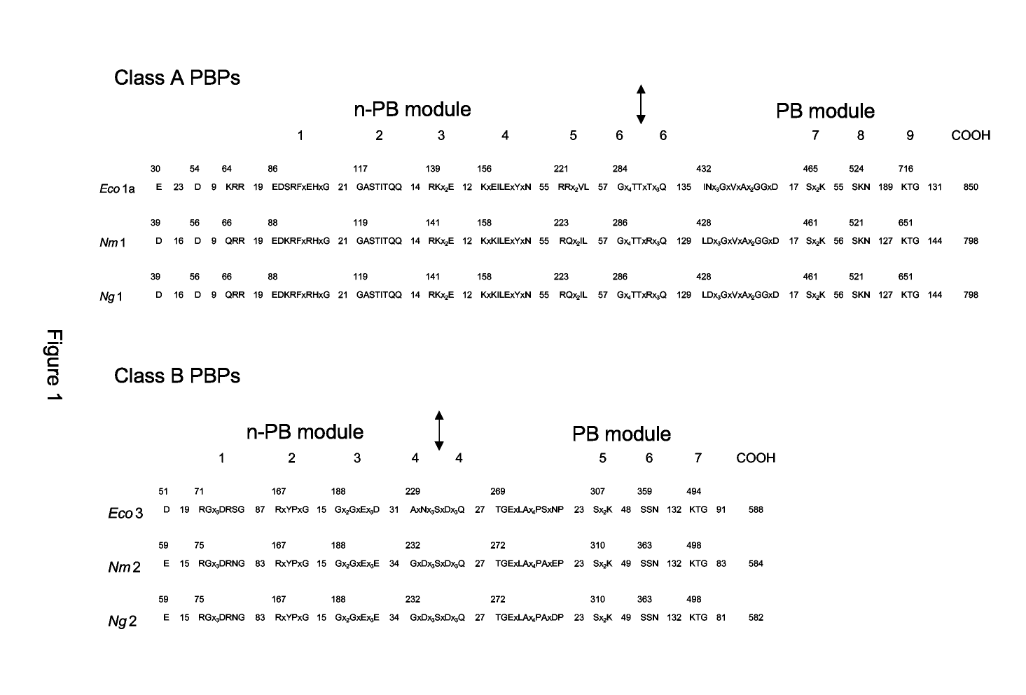

### EXAMPLE 1

### Production of Recombinant Neisseria meningitidis PBP2 Fragments Lacking the N-Terminal Transmembrane Domain

Modified penA genes lacking the sequence coding for the N-terminal transmembrane domain of PBP2 from several meningococcal Pen^{S} and Pen^{I} isolates were cloned into the pET28b expression vector (Novagen, Nottingham, UK).

Escherichia coli BL21 pLysS DE3 strain was then transformed with recombinant plasmids and His₆-tag-PBP2s were overexpressed under the control of the T7 bacteriophage promoter.

Purification of the recombinant proteins was performed using a nickel nitrilotriacetic acid-agarose column (Qiagen, Duren-Germany) as previously reported (Antignac, A., et al. 2003. J Biol Chem 278:31529-35).

After SDS-PAGE analysis the fractions containing purified PBP2 were pooled, dialyzed against phosphate-buffered saline, adjusted to 20% glycerol (v\v), and stored at −20° C.

Features of all Neisseria meningitidis strains for which a recombinant Neisseria meningitidis PBP2 fragment lacking the N-terminal transmembrane domain was produced are listed in following Table 3.

TABLE 3
Neisseria meningitidis strains for which a recombinant
Neisseria meningitidis PBP2 fragment lacking
the N-terminal transmembrane domain was produced.

| | | | | MIC (μg/ml) |
|---|---|---|---|---|
| Strain | Serogroup | Serotype | Serosubtype^{a} | Penicillin G^{b} |
| LNP8013 | C | NT | NST | 0.094 |
| LNP16454 | C | 2a | P1-5 | 1 |
| LNP16504 | B | NT | NST | 0.25 |
| LNP17041 | B | 1 | NST | 0.5 |
| LNP17723 | C | 2a | P1-5 | 0.75 |
| TR TH-41 | C | NT | NST | 0.5 |
| TR 16454 | C | NT | NST | 0.5 |
| TR 16504 | C | NT | NST | 0.38 |

^{a}NT, nontypable; NST, nonsubtypable
^{b}MICs: susceptible < 0.125 μg/ml; resistant > 1 μg/ml

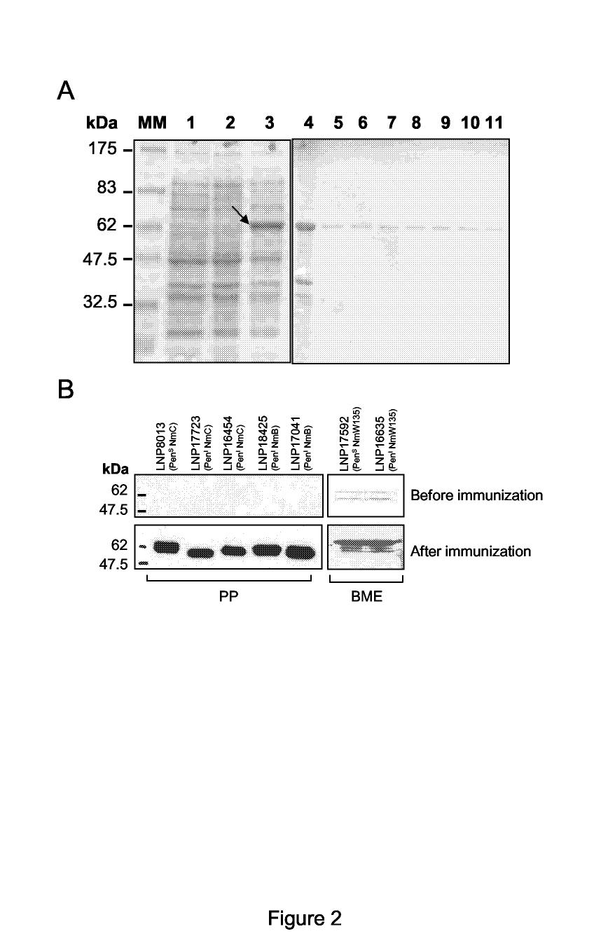

FIG. 2A shows the analysis of the different purification steps for a recombinant Neisseria meningitidis PBP2 fragment lacking the N-terminal transmembrane domain of penicillin susceptible Neisseria meningitidis serogroup C strain LNP8013.

### EXAMPLE 2

### Binding Capacities of a Rabbit Anti-PBP2 Serum Obtained after Immunization with the Recombinant Neisseria meningitidis PBP2 Lacking the N-Terminal Transmembrane Domain

The anti-PBP2 serum was obtained after immunization of rabbits with the purified recombinant His₆-tag-PBP2 lacking the N-terminal transmembrane domain from the penicillin susceptible serogroup C strain LNP8013 (FIG. 2A).

For PBP2 immunization, two 3-kg female rabbits were injected subcutaneously with 3 μg of the purified PBP2 supplemented with Freund complete adjuvant (Difco, Mass.) on day 0 and with Freund incomplete adjuvant on days 7 and 21. Pre-immune sera were obtained on day 0 before immunization and immune sera were collected on day 28.

To test the specificity of the rabbit anti-PBP2 sera, a western blot against penA^{S} and penA^{I} encoded PBP2s from meningococci belonging to different serogroups and genetic lineages was performed. Five thousand-fold dilution of sera permitted to show specific recognition by only immune sera.

The specificity of the rabbit anti-PBP2 sera was tested on the purified recombinant Neisseria meningitidis PBP2 fragments lacking the N-terminal transmembrane domain of penicillin described in Example 1, as well as on bacterial membrane extracts of two other Neisseria meningitidis strains belonging to serogroup W135 (strains LNP17592 and LNP16635).

The results show that the tested rabbit anti-PBP2 sera recognize PBP2s from several different meningococcal isolates belonging to serogroups B, C or W135, independently of the level of penicillin susceptibility of the different strains (FIG. 2B).

This confirms that the purified PBP2 protein is immunogenic in rabbit and that anti-PBP2 antibodies recognize PBP2s from several different meningococcal isolates, suggesting that they might have a protective effect if injected to a infected subject during Neisseria meningitidis infection by many different Neisseria meningitidis strains, including serogroup B strains.

### EXAMPLE 3

### Immunogenicity of PBP2 during Natural Meningococcal Infection

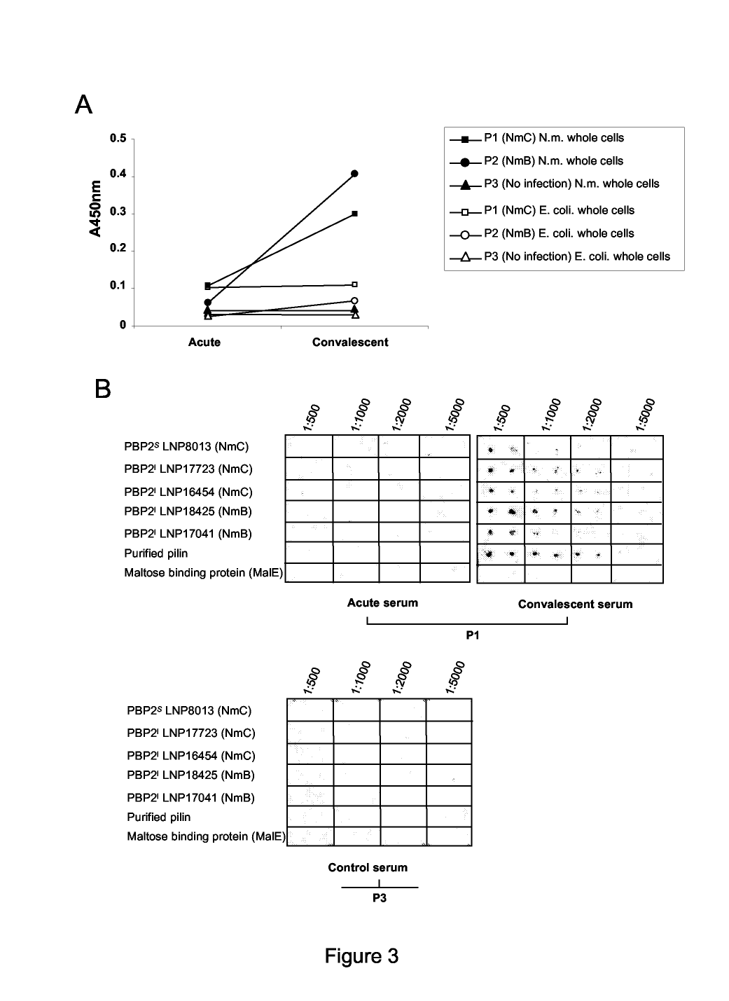

Purified recombinant PBP2s corresponding to meningococcal isolates belonging to different genetic lineages, different serogroups (B and C), and various susceptibility to penicillin G, were spotted on nitrocellulose membrane and immuno-detected using a pair of sera from a patient (P1) with confirmed meningococcal infection (positive culture for a serogroup C Neisseiria meningitidis). The paired sera consisted of an acute serum obtained at admission to hospital and a convalescent serum obtained 10 days later. Serial serum dilutions were tested and IgG response was detected using a goat anti-human IgG (CALTAG laboratories, Burlingame Calif.). An IgG signal was only detected using convalescent serum against all tested penA^{S} and penA^{I} encoded PBP2s (FIG. 3B).

Similar results were obtained when PBP2s were immunodetected with a pair of sera obtained from another patient (P2) with a confirmed serogroup B meningococcal infection. No signal was detected with both sera against an irrelevant protein (maltose binding protein, MalE of E. coli) while a positive signal was observed against a major meningococcal immunogenic protein (pilin) with the convalescent serum (FIG. 3B).

This increase in anti-PBP2 immunoblotting paralleled the immune response of patients sera (dilution 1:1,000) tested by ELISA against Neisseiria meningitidis whole cells (FIG. 3A).

No enhancement of signal was observed with a couple of control sera obtained from a patient (P3) with no meningococcal infection (FIG. 3A). These data confirm the specificity of the immune response against PBP2 during natural meningococcal infection, suggesting that Neisseria meningitidis PBP2 should be a convenient target for protective antibodies during Neisseria meningitidis infection.

### EXAMPLE 4

### Passive Protection Studies against Meningococcal Bacteremia in Mice Challenged Intraperitoneally

4.1 Passive Protection using Polyclonal Anti-PBP2 IgG

Next, the ability of a rabbit anti-PBP2 IgG to confer passive protection against Neisseiria meningitidis bacteremia in a murine intraperitoneal (i.p.) model of infection was tested.

Anti-PBP2 IgG was obtained by purification of total IgG from one rabbit immune serum after immunization by the purified recombinant His₆-tag-PBP2 lacking the N-terminal transmembrane domain from the penicillin susceptible serogroup C strain LNP8013. Total IgG were first precipitated with 33% (NH₄)₂SO₄ and dialyzed against phosphate saline buffer 17.5 mM, pH 6.3. This fraction was adsorbed on a cianogen bromide activated Sepharose 4B column (Pharmacia, Sweden) previously coated with a bacterial extract of BL21 pLysS DE3 E. coli harboring the pET28B expression vector. Protein concentration in the eluted fraction was determined by absorbance at 280 nm.

For passive protection experiments, all tested meningococcal strains were passaged in mice to maintain virulence and stored at −70° C. in GC-broth with 30% of glycerol. They were grown overnight at 37° C. in 5% CO₂ on GCB agar plates and were then inoculated into BHI broth. Bacteria were grown for 4 h to reach the exponential phase and adjusted at 10⁶ cfu/ml.

Five-week-old female BALB/c mice (Harlan, France) were treated at time 0 with an intravenous administration of 30 μg, 3 μg and 0.3 μg of purified anti-PBP2 rabbit IgG. These doses were calculated to roughly achieve in mice a concentration (0.3 μg/ml to 30 μg/ml) of specific IgGs similar to that expected in humans (about 0.3 to 0.5% of total IgGs). For each experiment, control mice were injected with PBS or with a purified rabbit IgG against the meningococcal cytoplasmic (thus not accessible to antibodies) regulatory protein, CrgA (Deghmane, A. E., and M. K. Taha. 2003. Mol Microbiol 47:135-43).

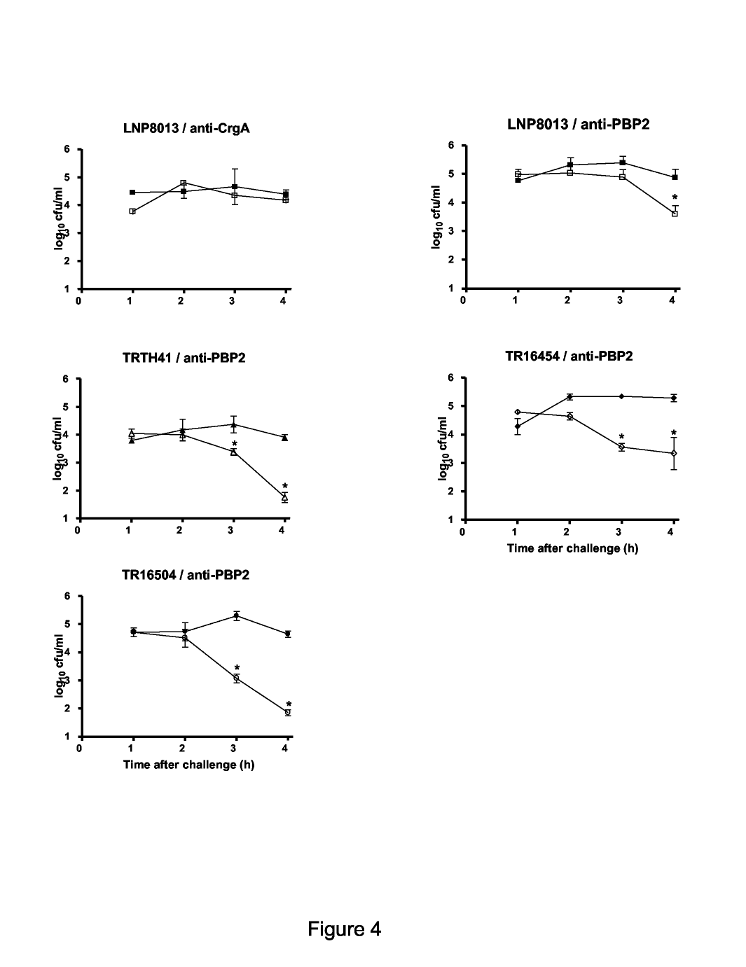

Two hours later mice were challenged i.p. with 0.5 ml (5×105 cfu) of the serogroup C Neisseiria meningitidis strain LNP8013, a penicillin susceptible strain harboring a wild type penA^{S} allele. Blood cfu counts from three mice and from two independent experiments were performed 1 h, 2h, 3h and 4h after the bacterial challenge. There was no significant difference in the level of bacteremia after 1 and 2 hours post challenge among mice (FIG. 4). However anti-PBP2 rabbit IgG-injected mice showed a reduction in bacteremia after 3 and 4 hours when compared to controls and this reduction was dose-dependent for the three used doses (30 μg, 3 μg and 0.3 μg) (FIG. 4). No protection was observed for the negative controls, i.e. mice injected with PBS or with purified rabbit IgG against the meningococcal cytoplasmic regulatory protein, CrgA (FIG. 3) (Deghmane, A. E., and M. K. Taha. 2003. Mol Microbiol 47:135-43).

Next, the ability of the anti-PBP2 IgG to passively protect against i.p. challenge with three LNP8013 isogenic mutants with reduced susceptibility to penicillin was analyzed. All these strains have alterations within the 3′ transpeptidase-encoding region of the penA gene. The TR-TH41, TR16454 and TR16504 strains were obtained by transformation of LNP8013 with the PCR amplified penA allele from three different meningococcal strains (TH-41, LNP16454 and LNP16504, respectively) (Antignac, A., et al. 2003. J Biol Chem 278:31529-35). As observed in FIG. 4, the anti-PBP2 IgG reduced bacteremia after challenge with all three penA mutants, despite alterations in the PBP2 protein they express.

These results demonstrate that an injection of antibodies binding a particular Neisseria meningitidis strain PBP2 protein is sufficient to induce a protection of a mouse infected by this particular strain, but also by mutant Neisseria meningitidis strains. It thus suggests that the injection of antibodies binding a particular Neisseria meningitidis strain PBP2 protein should be sufficient to induce a protection against infection by many distinct Neisseria meningitidis strains, including Neisseria meningitidis strains of other serogroups.

4.2 Specificity of Passive Protection using Polyclonal Anti-PBP2 IgG

To further confirm the specificity of the passive protection observed using the purified anti-PBP2 IgGs, a set of experiments was performed where the bacterial challenge was performed 2 h after injection of purified anti-PBP2 IgG alone or with the purified recombinant PBP2 fragment lacking the N-terminal transmembrane domain used for anti-PBP2 IgG production.

More precisely, five-week-old female BALB/c mice (Janvier, France) were injected at time 0 with a solution containing 30 μg of purified rabbit anti-PBP2 IgG alone or with 20 μg of purified recombinant His₆-tag-PBP2 lacking the N-terminal transmembrane domain from the penicillin susceptible serogroup C strain LNP8013. Two hours later, mice were challenged i.p. with 3×10⁶ cfu of the bacterial suspension in 0.5 mL of PBS. Blood cfu counts from four mice were performed 4 h after the bacterial challenge in two independent experiments.

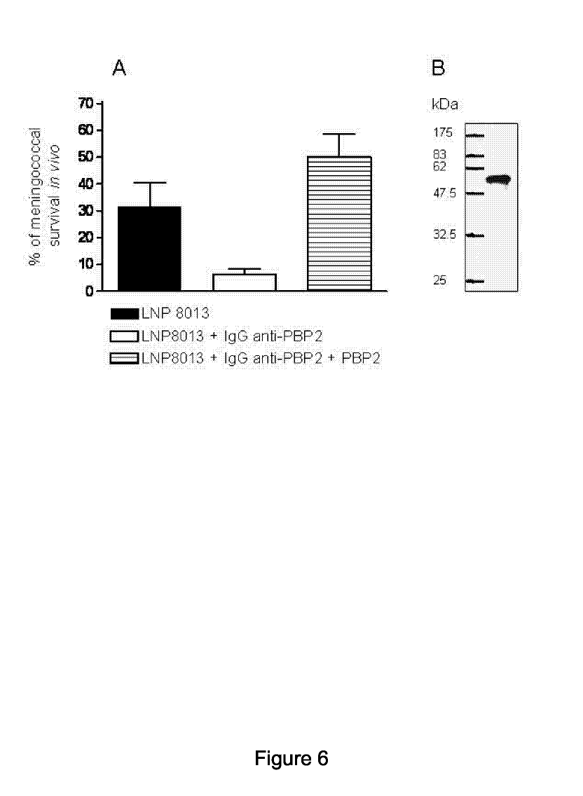

A significant higher level (P=0.00012) of bacteraemia was observed in mice treated with both purified anti-PBP2 IgG and purified PBP2 recombinant fragment lacking the N-terminal transmembrane domain compared to mice treated with purified anti-PBP2 IgG alone (see FIG. 6A). In addition, Anti-PBP2 IgG recognized only one band in the purified preparation of the recombinant PBP2 fragment (FIG. 6B). Altogether, these results strongly suggest that the passive protection conferred by the polyclonal anti-PBP2 IgG is due to the specific recognition of- and binding to the meningococcal PBP2.

### EXAMPLE 5

### Vaccination of Mice with a Fragment of Neisseria meningitidis PBP2 Protein

### Methods

Five female BALB/c mice that were 6 weeks old were immunised with 12 μg of the purified recombinant His₆-tag-PBP2 lacking the N-terminal transmembrane domain from the penicillin susceptible serogroup C strain LNP8013 (PBP2C^{S}). Four control mice were injected with saline solution. Injections were performed subcutaneously on days 0, 7 and 14 using complete Freund's adjuvant (Difco, Detroit, Mich., USA) for the first dose and incomplete Freund's adjuvant for the second one. On day 28 mice were bled to test immune response by ELISA using 96-well PBP2C^{S} coated plates with 1 μg of the purified PBP2. Intraperitoneal (i.p.) challenge with 5×10⁵ cfu of strain LNP8013 (Pen^{S}) was then performed. Blood colony forming units (cfu) counts were performed on GCB plates 3h after the bacterial challenge.

### Results

A good level of anti-PBP2 antibodies titres was observed by ELISA as compared to control mice. No difference in antibody titre was observed among vaccinated mice.

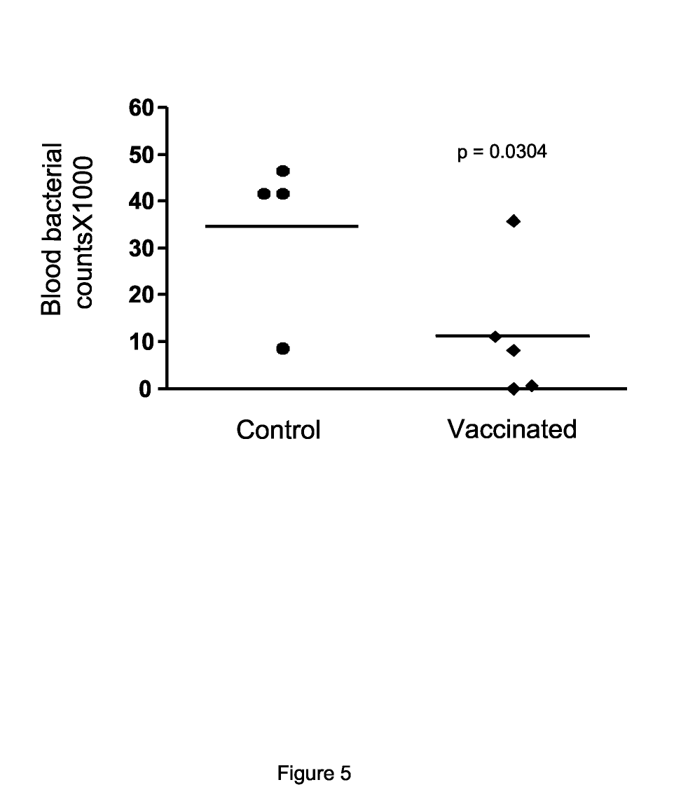

Vaccinated mice showed significant lower cfu counts in blood when compared to control mice (P=0.0304) (FIG. 5). These results suggest that purified recombinant His₆-tag-PBP2 lacking the N-terminal transmembrane domain from the penicillin susceptible serogroup C strain LNP8013 (PBP2C^{S} is immunogenic and is able to induce a protective response against Neisseiria meningitidis.

Overall, the results described in the previous examples clearly show that Neisseria meningitidis PBP2 protein is immunogenic during normal human Neisseria meningitidis infection and that immunization of mice with a PBP2 fragment induces anti-PBP2 antibodies.

In addition, such an immunization induces protective immunity in vaccinated mice.

Most of all, despite the high genetic variability of Neisseria meningitidis PBP2 protein, the results show that antibodies directed against a particular Neisseria meningitidis PBP2 protein are able to bind Neisseria meningitidis PBP2 proteins of other different Neisseria meningitidis strains, and that such antibodies confer a protection if injected during a Neisseria meningitidis infection.

The ability of antibodies directed against a particular Neisseria meningitidis PBP2 protein to bind Neisseria meningitidis PBP2 proteins of other different Neisseria meningitidis strains is probably linked to the presence of the conserved amino acid motifs (Sx₂K, SxN, and KTG) in the PB module. These motifs being highly conserved among PBP proteins, either among mutants of a particular PBP protein or even among different bacteria groups, this strategy should thus be also applicable to vaccinates mammals against infection by bacterial strains of distinct species expressing a variant of a Neisseria meningitidis PBP protein.

PBP proteins expressed by Neisseria meningitidis could thus be used for vaccination against Neisseria meningitidis and/or bacterial strains of dictinct species expressing variants of Neisseria meningitidis PBP proteins. Antibodies directed against Neisseria meningitidis PBP proteins could be used for the therapeutic treatment of individuals infected by Neisseria meningitidis and/or bacterial strains of distinct species expressing variants of Neisseria meningitidis PBP proteins. To increase the protection effect of PBP proteins or antibodies administrations, a combination of several PBP proteins or antibodies may be used.

### EXAMPLE 6

### Preparation of Monoclonal Antibodies Directed Against Neisseria meningitidis PBP2 protein

### Methods

Purified recombinant His₆-tag-PBP2 lacking the N-terminal transmembrane domain from the penicillin susceptible serogroup C strain LNP8013 (purified from clone DH5pAA2 deposited at CNCM on May 3, 2005 under number I-3422) was used for the production of specific monoclonal antibodies.

Swiss mice (Janvier) were injected with PBP2 at day 0, 7 and 21. Responses were then checked against the purified PBP2CS. Responder mice were kept during four month without further immunisation. A booster injection was given with the same dose of PBP2 recombinant fragment 3 days before the fusion.

The mouse that gave the highest antibody titers was sacrificed and its spleen was collected. Spleen cells from immunised mouse and the mouse myeloma cells P3U1 were mixed at a 4:1 ratio, pelleted and fused by the addition of 0.8 ml of 45% polyethylene glycol 1000.

The resulting fusion cells were grown on 24-well plates under 5% carbon dioxide at 37° C. Stable hybrid cell lines were selected using hypoxanthine-azaserin medium.

Two weeks after fusion, hybridoma supernatants were screened for the presence of specific anti-PBP2 antibodies by ELISA. Cells from positive wells were cloned by limiting dilution.

The classes and subclasses of the selected monoclonal antibodies were determined by ELISA using peroxidase-conjugated immunoglobulins specific for different subclasses.

Hybridoma cells were expanded and ascite production was performed by injecting 5×10⁶ cells intraperitoneally into Nude mice (NMRI-nu, Janvier).

### Results

Using the above described protocol, three hybridoma producing monoclonal antibodies directed against Neisseria meningitidis PBP2 protein were obtained.

These 3 hybridoma, denominated D2.4, D13.6 and 14.37, were deposited at CNCM (25-28 rue du Dr Roux. 75015 Paris. France) on May 22, 2006 under numbers I-3609, I-3610 and I-3611 respectively.

All three monoclonal antibodies produced from said hybridomas were IgG antibodies.

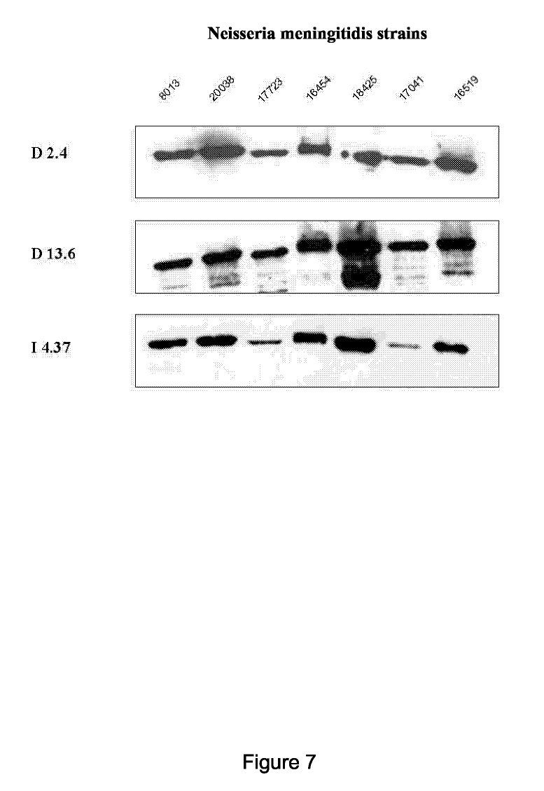

The binding capacities of said three anti-PBP2 monoclonal antibodies (D2.4, D13.6 and 14.37) were tested by western blot. All three monoclonal antibodies recognized purified recombinants PBP2s from several different meningococcal strains belonging to serogroups B, C or W135 and with different levels of susceptibility to penicillin (see FIG. 7).

1. A penicillin-binding protein expressed by Neisseria meningitidis, or a fragment, or a variant, or a variant fragment thereof, as a medicament.

2. A nucleic acid encoding at least one penicillin-binding protein expressed by Neisseria meningitidis, or a fragment, or a variant, or a variant fragment thereof, as a medicament.

3. An antibody or an antibody fragment directed against a penicillin-binding protein expressed by Neisseria meningitidis, or a variant, or a fragment, or a variant fragment thereof, as a medicament.

4. A medicament according to claim 1, wherein said penicillin-binding protein is Neisseria meningitidis PBP2.

5. A medicament according to claim 4, wherein said penicillin-binding protein fragment is a fragment of Neisseria meningitidis PBP2 lacking the N-terminal transmembrane domain.

6. A medicament according to claim 5, wherein said fragment of Neisseria meningitidis PBP2 lacking the N-terminal transmembrane domain is purified from clone DH5pAA2.

7. A medicament according to claim 1, wherein said variant is a variant of a Neisseria meningitidis PBP expressed by Neisseria gonorrhoeae or Streptococcus pneumoniae.

8. A medicament according to claim 7, wherein said variant is Neisseria gonorrhoeae PBP2 or Streptococcus pneumoniae PBP2X.

9. A pharmaceutical composition comprising at least one penicillin-binding protein, or fragment, or variant, or variant fragment thereof; of claim 1, with a pharmaceutically acceptable carrier or excipient.

10. A method for vaccination against infection by a bacterial strain expressing a Neisseria meningitidis PBP protein or a variant thereof, comprising administering a pharmaceutical composition comprising at least one penicillin-binding protein or fragment, or variant, or variant fragment thereof, according to claim 1, to a mammal.

11. Use of A method for vaccination against infection by a bacterial strain expressing a Neisseria meningitidis PBP protein or a variant thereof, comprising administering a pharmaceutical composition comprising at least one nucleic acid according to claim 2, to a mammal.

12. A method for vaccination against infection by a bacterial strain expressing a Neisseria meningitidis PBP protein or a variant thereof comprising administering a pharmaceutical composition comprising at least one antibody or antibody fragment according to claim 3, to a mammal a human being.

13-14. (canceled)

15. The method of claim 10, wherein said infectious bacterial strain is Neisseria gonorrhoeae or Streptococcus pneumoniae.

16-17. (canceled)

18. The method of claim 10, wherein the pharmaceutical composition comprises an antibiotic.

19. The method of claim 18, wherein said antibiotic is a β-lactam antibiotic.

20. (canceled)

21. A method of in vitro detection from a biological sample of a mammal, of antibodies directed against Neisseria meningitidis infection, comprising:
contacting said biological sample with at least one Neisseria meningitidis penicillin-binding protein, or fragment, or variant or variant fragment thereof according to claim 1, and
detecting the presence of complexes between said at least one Neisseria meningitidis penicillin-binding protein, or fragment, or variant or variant fragment thereof and an antibody of said biological sample.

22. (canceled)

23. A method of in vitro diagnosis from a biological sample of a mammal, of Neisseria meningitidis infection, comprising:
contacting said biological sample with at least one antibody or antibody fragment according to claim 3, and
detecting the presence of complexes between said at least one antibody or antibody fragment and a Neisseria meningitidis penicillin-binding protein, or fragment thereof from said biological sample.

24. A monoclonal antibody directed against Neisseria meningitidis PBP2 protein selected from the group consisting of monoclonal antibodies produced from hybridomas D2.4, D13.6 and 14.37.


SEQUENCE LISTING (13 sequences)

SEQ ID NO: 1 - protein, 798 aa, Neisseria meningitidis
  MISC_FEATURE: PBP1 protein of Neisseria meningitidis (MC58)
MIKKILTTCFGLVFGFCVFGVGLVAIAILVTYPKLPSLDSLQHYQPKMPLTIYSADGEVIGMYGEQRREFTKIGDFPEVLRNAVIAAEDKRFYRHWGVDVWGVARAAVGNVVSGSVQSGASTITQQVAKNFYLSSEKTFTRKFNEVLLAYKIEQSLSKDKILELYFNQIYLGQRAYGFASAAQIYFNKNVRDLTLAEAAMLAGLPKAPSAYNPIVNPERAKLRQKYILNNMLEEKMITVQQRDQALNEELHYERFVRKIDQSALYVAEMVRQELYEKYGEDAYTQGFKVYTTVRADHQKVATEALRKALRNFDRGSSYRGAENYIDLSKSEDVEETVSQYLSGLYTVDKMVPAVVLDVTKKKNVVIQLPGGRRVTLDRRALGFAARAVNNEKMGEDRIRRGAVIRVKNNGGRWAVVQEPLLQGALVSLDAKTGAVRALVGGYDFHSKTFNRAVQAMRQPGSTFKPFVYSAALSKGMTASTVVNDAPISLPGKGPNGSVWTPKNSDGRYSGYITLRQALTASKNMVSIRILMSIGVGYAQQYIRRFGFRSSELPASLSMALGTGETTPLKVAEAYSVFANGGYRVSSHVIDKIYDRDGRLRAQMQPLVAGQNAPQAIDPRNAYIMYKIMQDVVRVGTARGAAALGRTDIAGKTGTTNDNKDAWFVGFNPDVVTAVYIGFDKPKSMGRVGYGGTIAVPVWVDYMRFALKGKQGKGMKMPEGVVSSNGEYYMKERMVTDPGLTLDNSGIAPQPSRRAKEDDGGAAEGGRQAADDEVRQDMQETPVLPSNTGSKQQQLDSLF

SEQ ID NO: 2 - protein, 798 aa, Neisseria meningitidis; identical to SEQ ID NO: 1
  MISC_FEATURE: PBR1 protein of Neisseria meningitidis (Z2491)

SEQ ID NO: 3 - protein, 581 aa, Neisseria meningitidis
  misc_feature: PBP2 protein of Neisseria meningitidis (MC58)
MLIKSEYKPRMLPKEEQVKKPMTSNGRISFVLMAIAVLFAGLIARGLYLQTVTYNFLKEQGDNRIVRTQTLPATRGTVSDRNGAVLALSAPTESLFAVPKEMKEMPSAAQLERLSELVDVPVDVLRNKLEQKGKSFIWIKRQLDPKVAEEVKALGLENFVFEKELKRHYPMGNLFAHVIGFTDIDGKGQEGLELSLEDSLHGEDGAEVVLRDRQGNIVDSLDSPRNKAPKNGKDIILSLDQRIQTLAYEELNKAVEYHQAKAGTVVVLDARTGEILALANTPAYDPNRPGRADSEQRRNRAVTDMIEPGSAIKPFVIAKALDAGKTDLNERLNTQPYKIGPSPVRDTHVYPSLDVRGIMQKSSNVGTSKLSARFGAEEMYDFYHELGIGVRMHSGFPGETAGLLRNWRRWRPIEQATMSFGYGLQLSLLQLARAYTALTHDGVLLPVSFEKQAVAPQGKRIFKESTAREVRNLMVSVTEPGGTGTAGAVDGFDVGAKTGTARKFVNGRYADNKHIATFIGFAPAKNPRVIVAVTIDEPTAHGYYGGVVAGPPFKKIMGGSLNILGISPTKPLTAAAVKTPS

SEQ ID NO: 4 - protein, 581 aa, Neisseria meningitidis; identical to SEQ ID NO: 3
  MISC_FEATURE: PBP2 protein of Neisseria meningitidis (Z2491))

SEQ ID NO: 5 - protein, 469 aa, Neisseria meningitidis
  MISC_FEATURE: PBP3 protein of Neisseria meningitidis (MC58)
MNFPKTAASLLLLLASLAAHALDTGRIPQNEIAVYVQELDSGKVIIDHRSDVPVNPASTMKLVTAFAAFKTFGSNYRWATEFKSNGTVNDGTLDGNLYWAGSGDPVFNQENLLDAQKQLREQGILNITGHLMLDHSLWGEVGSPDDFEADSGSPFMTPPNPTMLSAGMVMVRAERNAAGSTDILTDPPLPHIFAQNNLKITASQAACPSIKKLMRASFSDNTLKLRGNIPESCLGKPVGVRMFALDELIRQSFTNHWLLGGGRISDGIGIADTPEGAQTLAVAHAKPMKEILTDMNKRSDNLIARSVFLKLGGDGKLPAVSEQAASAVRRELAVSGIDVADLVLENGSGLSRKERVTARMMAQMLETAYFSPFAQDFIDTLPIAGTDGTLRNRFKQSGGLLRLKTGTLNNVRALAGYWLGDKPMAVVVIINSGRAVSLLPDLDNFVANNIISGGDGWLDAKLMCKERRA

SEQ ID NO: 6 - protein, 312 aa, Neisseria meningitidis
  MISC_FEATURE: PBP4 protein of Neisseria meningitidis (MC58)
MSIHTLKRLPSSLLLGLCLSLPSAHLFADNDILGQFLEQNMLTSSDPIEIFAESTIHPTNTQAITGGLILSSQSALVVNNKTGQILYQKNADRIMPIASISKLMSAMVVLDANLDMNETVTITPDEIDRIKGTGSRLAIGTALTRKKLLHLSLMSSENRATHALGRTYPGGMGAFVAAMNRKAQSLGMYGSRFYEPTGLNFQNVSTAKDLSLMVNAAAQYPQIRTNSTSNYASVQTKNGQQNYKNSNALVREGMWNIELQKTGYIREAGRSMVVKANIQNQPVTIVLLNSPTSATRVNDARKIESWMLQQRS

SEQ ID NO: 7 - protein, 312 aa, Neisseria meningitidis
  MISC_FEATURE: PBP4 protein of Neisseria meningitidis (Z2491)
MSIHTLKRLPSSLLLGLCLSLPSAHLFADNDILGQFLEQNMLTSSDPIEIFAESTIHPTNTQAITGGLILSSQSALVVNNKTGQILYQKNADRIMPIASISKLMSAMVVLDANLDMNETVTITPDEIDRIKGTGSRLAIGTALTRKELLHLSLMSSENRATHALGRTYPGGMGAFVAAMNRKAQSLGMYGSRFYEPTGLNFQNVSTAKDLSLMVNAAAQYPQIRTNSTSNYASVQTKNGQQNYKNSNALVREGMWNIELQKTGYIREAGRSMVVKANIQNQPVTIVLLNSPTSATRVNDARKIESWMLQQRS

SEQ ID NO: 8 - protein, 389 aa, Neisseria meningitidis
  MISC_FEATURE: PBP5 protein of Neisseria meningitidis (MC58)
MTAHKILPVLLSIILGVSHATAASPAPNRPTVHAAPTFQTPETLTAAHIVIDLQSKQILSAKNINTPVEPAALTQLMTAYLVFKNMKSGNIQSEENLKIPESAWASEGSRMFVRPGDTVSTDKLLKGMIALSANDAALTLAGRLGNGSIENFVQQMNKEARRLGMKNTVFKNPTGLSREGQVSTAKDLALLSEALMRDFPEYYPLFSIKSFKFKNIEQNNRNILLYRDNNVNGLKAGHTESGGYNLAVSYSGNGRHILVITLGSESAETRASDNSKLLNWALQAFDTPKIYPKGKTVAQIQISGGSKKTVRAGFLKEAYITLPHKEAKMAEQILETIQPIPAPVKKGQILGKIKIRQNGYTIAEKEIVALENVKKRSRWQRLWACLTGQ

SEQ ID NO: 9 - protein, 798 aa, Neisseria meningitidis
  MISC_FEATURE: PBP1 protein of Neisseria gonorrhoeae (FA 1090)
MIKKILTTCFGLFFGFCVFGVGLVAIAILVTYPKLPSLDSLQHYQPKMPLTIYSADGEVIGMYGEQRREFTKIGDFPEVLRNAVIAAEDKRFYRHWGVDVWGVARAAVGNVVSGSVQSGASTITQQVAKNFYLSSEKTFTRKFNEVLLAYKIEQSLSKDKILELYFNQIYLGQRAYGFASAAQIYFNKNVRDLTLAEAAMLAGLPKAPSAYNPIVNPERAKLRQKYILNNMLEEKMITVQQRDQALNEELHYERFVRKIDQSALYVAEMVRRELYEKYGEDAYTQGFKVYTTVRTDHQKAATEALRKALRNFDRGSSYRGAENYIDLSKSEDVEETVSQYLSGLYTVDKMVPAVVLDVTKKKNVVIQLPGGRRVALDRRALGFAARAVDNEKMGEDRIRRGAVIRVKNNGGRWAVVQEPLLQGALVSLDAKTGAVRALVGGYDFHSKTFNRAVQAMRQPGSTFKPFVYSAALSKGMTASTVVNDAPISLPGKGPNGSVWTPKNSDGRYSGYITLRQALTASKNMVSIRILMSIGVGYAQQYIRRFGFRPSELPASLSMALGTGETTPLKVAEAYSVFANGGYRVSSHVIDKIYDRDGRLRAQMQPLVAGQNAPQAIDPRNAYIMYKIMQDVVRVGTARGAAALGRTDIAGKTGTTNDNKDAWFVGFNPDVVTAVYIGFDKPKSMGRAGYGGTIAVPVWVDYMRFALKGKQGKGMKMPEGVVSSNGEYYMKERMVTDPGLMLDNSGIAPQPSRRAKEDDEAAVENEQQGRSDETRQDVQETPVLPSNTDSKQQQLDSLF

SEQ ID NO: 10 - protein, 821 aa, Neisseria meningitidis
  misc_feature: PBP1b protein of Streptococcus pneumoniae (R6)
MQNQLNELKRKMLEFFQQKQKNKKSARPGKKGSSTKKSKTLDKSVIFPAILLSIKALFNLLFVLGFLGGMLGAGIALGYGVALFDKVRVPQTEELVNQVKDISSISEITYSDGTVIASIESDLLRTSISSEQISENLKKAIIATEDEHFKEHKGVVPKAVIRATLGKFVGLGSSSGGSTLTQQLIKQQVVGDAPTLARKAAEIVDALALERAMNKDEILTTYLNVAPFGRNNKGQNIAGARQAAEGIFGVDASQLTVPQAAFLAGLPQSPITYSPYENTGELKSDEDLEIGLRRAKAVLYSMYRTGALSKDEYSQYKDYDLKQDFLPSGTVTGISRDYLYFTTLAEAQERMYDYLAQRDNVSAKELKNEATQKFYRDLAAKEIENGGYKITTTIDQKIHSAMQSAVADYGYLLDDGTGRVEVGNVLMDNQTGAILGFVGGRNYQENQNNHAFDTKRSPASTTKPLLAYGIAIDQGLMGSETILSNYPTNFANGNPIMYANSKGTGMMTLGEALNYSWNIPAYWTYRMLREKGVDVKGYMEKMGYEIPEYGIESLPMGGGIEVTVAQHTNGYQTLANNGVYHQKHVISKIEAADGRVVYEYQDKPVQVYSKATATIMQGLLREVLSSRVTTTFKSNLTSLNPTLANADWIGKTGTTNQDENMWLMLSTPRLTLGGWIGHDDNHSLSRRAGYSNNSNYMAHLVNAIQQASPSIWGNERFALDPSVVKSEVLKSTGQKPEKVSVEGKEVEVTGSTVTSYWANKSGAPATSYRFAIGGSDADYQNAWSSIVGSLPTPSSSSSSSSSSSDSSNSSTTRPSSSRARR

SEQ ID NO: 11 - protein, 731 aa, Neisseria meningitidis
  MISC_FEATURE: PBP2a protein of Streptococcus pneumoniae (R6)
MKLDKLFEKFLSLFKKETSELEDSDSTILRRSRSDRKKLAQVGPIRKFWRRYHLTKIILILGLSAGLLVGIYLFAVAKSTNVNDLQNALKTRTLIFDREEKEAGALSGQKGTYVELTDISKNLQNAVIATEDRSFYKNDGINYGRFFLAIVTAGRSGGGSTITQQLAKNAYLSQDQTVERKAKEFFLALELSKKYSKEQILTMYLNNAYFGNGVWGVEDASKKYFGVSASEVSLDQAATLAGMLKGPELYNPLNSVEDSTNRRDTVLQNMVAAGYIDKNQETKAAEVDMTSQLHDKYEGKISDYRYPSYFDAVVNEAVSKYNLTEEEIVNNGYRIYTELDQNYQANMQIVYENTSLFPRAEDGTFAQSGSVALEPKTGGVRGVVGQVADNDKTGFRNFNYATQSKRSPGSTIKPLVVYTPAVEASWALNKQLDNHTMQYDSYKVDNYAGIKTSREVPMYQALAESLNLPAVATVNDLGVDKAFEAGEKFGLNMEKVDRVLGVALGSGVETNPLQMAQAYAAFANEGLMPEAHFISRIENASGQVIASHKNSQKRVIDKSVADKMTSMMLGTFTNGTGISSSPADYVMAGKTGTTEAVFNPEYTSDQWVIGYTPDVVISHWLGFPTTDENHYLAGSTSNGAAHVFRNIANTILPYTPGSTFTVENAYKQNGIAPANTKRQVQTNDNSQTDDNLSDIRGRAQSLVDEASRAISDAKIKEKAQTIWDSIVNLFR

SEQ ID NO: 12 - protein, 582 aa, Neisseria meningitidis
  MISC_FEATURE: PBP2 protein of Neisseria gonorrhoeae (FA 1090)
MLIKSEYKPRMLPKEEQVKKPMTSNGRISFVLMAMAVLFACLIARGLYLQTVTYNFLKEQGDNRIVRTQALPATRGTVSDRNGAVLALSAPTESLFAVPKDMKEMPSAAQLERLSELVDVPVDVLRNKLEQKGKSFIWIKRQLDPKVAEEVKALGLENFVFEKELKRHYPMGNLFAHVIGFTDIDGKGQEGLELSLEDSLYGEDGAEVVLRDRQGNIVDSLDSPRNKAPQNGKDIILSLDQRIQTLAYEELNKAVEYHQAKAGTVVVLDARTGEILALANTPAYDPNRPGRADSEQRRNRAVTDMIEPGSAIKPFVIAKALDAGKTDLNERLNTQPYKIGPSPVRDDTHVYPSLDVRGIMQKSSNVGTSKLSARFGAEEMYDFYHELGIGVRMHSGFPGETAGLLRNWRRWRPIEQATMSFGYGLQLSLLQLARAYTALTHDGVLLPLSFEKQAVAPQGKRIFKESTAREVRNLMVSVTEPGGTGTAGAVDGFDVGAKTGTARKFVNGRYADNKHVATFIGFAPAKNPRVIVAVTIDEPTAHGYYGGVVAGPPFKKIMGGSLNILGISPTKPLTAAAVKTPS

SEQ ID NO: 13 - protein, 750 aa, Neisseria meningitidis
  MISC_FEATURE: PBP2X protein of Streptococcus pneumoniae (R6)
MKWTKRVIRYATKNRKSPAENRRRVGKSLSLLSVFVFAIFLVNFAVIIGTGTRFGTDLAKEAKKVHQTTRTVPAKRGTIYDRNGVPIAEDATSYNVYAVIDENYKSATGKILYVEKTQFNKVAEVFHKYLDMEESYVREQLSQPNLKQVSFGAKGNGITYANMMSIKKELEAAEVKGIDFTTSPNRSYPNGQFASSFIGLAQLHENEDGSKSLLGTSGMESSLNSILAGTDGIITYEKDRLGNIVPGTEQVSQRTMDGKDVYTTISSPLQSFMETQMDAFQEKVKGKYMTATLVSAKTGEILATTQRPTFDADTKEGITEDFVWRDILYQSNYEPGSTMKVMMLAAAIDNNTFPGGEVFNSSELKIADATIRDWDVNEGLTGGRMMTFSQGFAHSSNVGMTLLEQKMGDATWLDYLNRFKFGVPTRFGLTDEYAGQLPADNIVNIAQSSFGQGISVTQTQMIRAFTAIANDGVMLEPKFISAIYDPNDQTARKSQKEIVGNPVSKDAASLTRTNMVLVGTDPVYGTMYNHSTGKPTVTVPGQNVALKSGTAQIADEKNGGYLVGLTDYIFSAVSMSPAENPDFILYVTVQQPEHYSGIQLGEFANPILERASAMKDSLNLQTTAKALEQVSQQSPYPMPSVKDISPGDLAEELRRNLVQPIVVGTGTKIKNSSAEEGKNLAPNQQVLILSDKAEEVPDMYGWTKETAETLAKWLNIELEFQGSGSTVQKQDVRANTAIKDIKKITLTLGD